(19) United States
(12) Patent Application Publication
(10) Pub. No.: US 2002/0082434 A1
(43) Pub. Date: Jun. 27, 2002

(54) Process for the production of sterols

(76) Inventors: Mehdi Bonakdar, Langenfeld (DE); Gerhard Wollmann, Hilden (DE); Bernhard Gutsche, Hilden (DE); Joerg Schwarzer, Hilden (DE)

Correspondence Address: COGNIS CORPORATION, 2500 RENAISSANCE BLVD., SUITE 200, GULPH MILLS, PA, 19406

(21) Appl. No.: 09/923,626

(22) Filed: Aug. 7, 2001

(30) Foreign Application Priority Data
Aug. 7, 2000 (DE) 100 38 456.0

(51) Int. Cl. C07J 9/00

(52) U.S. Cl. 552/545

(57) ABSTRACT

The invention relates to a process for the production of sterols from residues of the distillation of oils, characterized in that the partial glycerides are subjected to a first transesterification step and, after working up and removal of impurities, the sterol esters are subjected to a second transesterification step under more extreme conditions.

### BACKGROUND OF THE INVENTION

The production of sterols from distillates obtained in the deacidification of vegetable oils or from distillation residues accumulating in the production of methyl esters and more particularly in the production of methyl esters from crops for “biodiesel” applications is generally known.

One known process for the production of sterols has been described wherein a residue from the distillation of methyl esters essentially consisting of glycerides, sterols, sterol esters and tocopherols is transesterified with methanol in the presence of alkaline catalysts. However, the sterol yield is unsatisfactory. Although some improvements have been made, including the utilization and revaluation of residues from the production of fats and oils and their constituents, such improvements can involve costly, elaborate processes using ecologically harmful solvents. For example, in some processes readily volatile sterols from the distillation residues of fat and oil production and working up are isolated by transesterification and subsequent dissolution in aprotic organic solvents.

Accordingly, the problem addressed by the present invention was to produce sterols in high yields and high purity by an economic process that would avoid toxicologically and ecologically unsafe solvents and, at the same time, to utilize residues from the distillation of transesterified oils more economically.

### SUMMARY OF THE INVENTION

This invention relates generally to sterol production and more particularly to a process for the production of sterols from residues of the distillation of transesterified oils.

In order to be able to obtain sterols in pure form, they have to be converted from the esterified to the free state. Otherwise they are very difficult to separate from the components accompanying them. The conversion into free sterols may be carried out, for example, by hydrolysis, saponification or transesterification. The present invention is directed to the use of transesterification mechanisms.

The present invention includes a process for producing sterols, which comprises: (a) providing an oil distillation residue, said residue comprising sterol esters and partial glycerides; (b) transesterifying the partial glycerides with a lower alcohol in the presence of a basic catalyst under mild transesterification conditions to form fatty acid alkyl esters and glycerol; (c) removing excess lower alcohol, the basic catalyst, the glycerol and the fatty acid alkyl esters, to form a bottom product comprising the sterol esters; and (d) transesterifying the sterol esters at a temperature of from 90° C. to 145° C. and a pressure of from 2 to 10 bar for a period of from 4 to 10 hours to form free sterols.

A preferred embodiment of the present invention is directed to a process for the production of sterols from residues of the distillation of transesterified oils, characterized in that (a) the partial glycerides present in the mixture are transesterified with a lower alcohol in the presence of a basic catalyst for 5 to 20 minutes at temperatures of 115 to 145° C. and under a pressure of 2 to 10 bar, (b) the excess lower alcohol is distilled off from the reaction mixture after the transesterification, (c) the transesterification catalyst and the glycerol present, if any, are removed, (d) the fatty acid alkyl ester is distilled off from the mixture and (e) the sterol esters and residual partial glycerides present in the bottom product are converted into free sterols and fatty acid esters by further transesterification for 4 to 8 hours at temperatures of 90 to 145° C. and under a pressure of 2 to 10 bar.

It has been found that a process for the production of sterols can be made more economical and friendlier to the environment by combining two separate transesterification steps. In a first transesterification step, the mono-, di- and triglycerides are reacted with a lower alcohol in the presence of a basic catalyst. Under the mild conditions, the sterol esters remain predominantly bound and only a small amount of free sterols is formed (<1% by weight). After removal of the excess alcohol, transesterification catalyst and glycerol, the fatty acid esters are distilled, resulting in concentration of the sterol esters at the bottom of the column. The sterol esters are then split into the free sterols in a second transesterification step carried out under more extreme conditions. By virtue of the fact that the impurities are removed in this transesterification step and the sterol esters are present in concentrated form, the free sterols can be obtained under far more economic conditions. The first transesterification step proceeds very quickly and saves time so that it can be carried out in a simple tube reactor. By virtue of the reduced amount of starting products, a relatively small stirred reactor is sufficient for the second transesterification. In addition, the sterol yield can be increased if, during crystallization, part of the mother liquor is returned to the crystallization process after filtration of the crystal suspension.

The process is suitable for various starting mixtures and does not involve the use of toxicologically and ecologically unsafe solvents. High-quality yields are achieved. The better utilization of the distillation residues leads to an economic, ecologically safe process that is easy to carry out on an industrial scale.

### DETAILED DESCRIPTION OF THE INVENTION

### Residues from the Distillation of Transesterified Oils

Residues from the distillation of transesterified, more particularly non-refined oils with a residual acid value below 2 are used as raw materials for the production of sterols. These residues are preferably residues from coconut oil, from palm kernel oil, from palm oil, from sunflower oil and from rapeseed oil with acid values of 0 to 6 and contain mixtures of di- and triglycerides, methyl esters, sterol esters and wax esters, preferably 35 to 40% by weight triglycerides, 10 to 20% by weight diglycerides, 20 to 25% by weight fatty acid methyl esters, 10 to 12% by weight sterol esters, 3 to 4% by weight wax esters and small quantities of free sterols and monoglycerides.

### First Transesterification Step—Transesterification of the Partial Glycerides

In the “transesterification of the partial glycerides” process step, only the tri-, di and monoglycerides are reacted with short-chain alcohols to form fatty acid esters. The sterol esters remain substantially bound. Only a small amount of free sterols is formed. Methanol is preferably used as the alcohol.

The methanol is added in a quantity of 5 to 40% by weight and preferably 10 to 20% by weight, based on the transesterified oil distillation residue.

The reaction is preferably carried out over a period of 5 to 20 minutes and more particularly 8 to 15 minutes at a temperature of 115 to 145° C. and more particularly at a temperature of 120 to 130° C. A pressure of 2 to 10 bar is spontaneously established at these temperatures. Under these conditions, which correspond to a low-temperature transesterification, a catalyst has to be added. Any transesterification catalyst may be used as the catalyst. 30% methanolic sodium methylate solution is preferred and is used in a quantity of preferably 0.5 to 1.8% by weight and more preferably 1.0 to 1.5% by weight, based on the transesterified oil distillation residue. Suitable reactors are stirred batch autoclaves and continuous reactors such as, for example, turbulent flow tube reactors.

Alternatively to the low-pressure transesterification, the transesterification may also be carried out under pressure. In this case, the reaction is preferably carried out at 220 to 260° C. and under a pressure of 20 to 80 bar. In the case of residues accumulating in the distillation of oils transesterified under high pressures, there is no need to add a catalyst because the catalyst—generally soaps of divalent metals, such as Mn, Zn or Ca—is already present in a large excess. The pressure transesterification is preferred when the transesterified oil distillation residues have acid values above 1 and more particularly above 5. The advantage of pressure transesterification is particularly relevant when the acid value is reduced to below 5 and more particularly to below 1 by this process.

### Removal of the Excess Alcohol

In the “flashing of the excess alcohol” process step, the hot reaction mixture from the “transesterification of the partial glycerides” is expanded into a receiver, 55 to 85% of the excess alcohol distilling off. The system cools down considerably—to 75-85° C. where methanol is used. The residual alcohol still left in the reaction product is preferably not distilled off and serves as solubilizer in the following stage.

### Catalyst Precipitation and Removal

Catalysts are still present in the residues from the distillation of oils transesterified under pressure. These catalysts are preferably Zn soaps (2000-3500 ppm) although other soaps are also suitable. The distillation residues may also contain many other metals, such as Fe, Al or Na, in concentrations of up to 300 ppm and heavy metals, such as Pb, Cr or Ni, in concentrations of up to 20 ppm. Nonmetals, such as P, Si or S, are present in concentrations of up to 300 ppm.

The catalyst soaps and the other metal compounds are soluble in the reaction mixture from the “transesterification of the partial glycerides”. In order to be able to remove them, they are converted with acids into insoluble compounds and precipitated. Aqueous solutions of citric acid or phosphoric acid are preferably used as the acids. The quantity of acid used is preferably once to twice the molar concentration of metal. At the same time, the addition of acid neutralizes the Na methylate used in the low-pressure transesterification of the partial glycerides.

After precipitation, the metal-containing sludge precipitated is removed. It is preferably centrifuged. Phase separation improves if 15 to 30% of the excess alcohol remains in the product in the “flashing of the excess alcohol” process step.

Alternatively, the precipitated metals are adsorbed. Suitable adsorbers are amorphous silica gels charged with organic acids such as, for example, Trisyl types (Grace). Where the metals are removed by adsorption, all the alcohol may be removed in the preceding “flashing of the excess alcohol” process step.

In both alternative processes, residual metal contents of less than 1 ppm are achieved.

The catalyst-free product still contains excess alcohol and free glycerol. To prevent any back-reaction in the next “fatty acid ester distillation” stage, the free glycerol and the residual alcohol are removed from the catalyst-free product by decantation and, if necessary, are washed out with water. The product is then dried.

### Distillation of the Fatty Acid Alkyl Esters

To concentrate the sterol esters, the fatty acid esters are distilled off, for example in a thin-layer evaporator. Methyl esters are preferably distilled at temperatures of 170 to 200° C. and under pressures of 1 to 5 mbar. According to the invention, it was essentially only the partial glycerides that were transesterified in the “transesterification of the partial glycerides” process step. Since the sterols are still largely present as sterol esters, they are higher boiling and are not distilled off during distillation of the fatty acid esters. According to the invention, they remain entirely as a concentrated valuable product in the bottom fraction. In addition, other low-boiling components may be removed.

According to the invention, wax esters are also distilled off with the fatty acid esters and are subsequently separated from the fatty acid esters by winterizing. Sterol-free methyl or ethyl esters with a purity of more than 97% are obtained in this way.

### Second Transesterification Step—Transesterification of the Sterol Esters

The sterol esters are concentrated to more than 40% in the bottom product of the fatty acid ester distillation process. They are converted into free sterols by transesterification with a short-chain alcohol, preferably methanol, in the presence of a catalyst. Since the transesterification of sterol esters has to take place under more rigorous conditions than the transesterification of partial glycerides, larger quantities of alcohol and catalyst and longer reaction times are necessary.

The quantity of alcohol added is 40 to 80% by weight and preferably 50 to 60% by weight of the bottom product of the fatty acid ester distillation process. Where methanol is the transesterification reagent, 40 to 60% by weight of the bottom product of the fatty acid ester distillation process is used. Here, too, the catalyst may be any transesterification catalyst.

The reaction preferably takes place over a period of 4 to 10 hours and more particularly 5 to 8 hours at temperatures of 90 to 145° C. and more particularly 120 to 130° C. and under a pressure of 2 to 10 bar. Any low-pressure transesterification catalyst may be used as the catalyst. In a preferred embodiment, a 30% methanolic sodium methylate solution is used in a quantity of 1.8 to 6% by weight and more particularly 2 to 4% by weight, based on the bottom product of the fatty acid ester distillation. The reactor used may be, for example, a stirred batch autoclave.

Alternatively to low-pressure transesterification, the transe-sterification may again be carried out under elevated pressure. In this case, the reaction takes place over a period of 4 to 8 hours at 200 to 260° C. and under a pressure of 20 to 80 bar. Any high-temperature transesterification catalyst may be used as the catalyst. Zn or Ca soap is preferably used.

The free sterols produced may then be purified by crystallization. However, the transesterified oil distillation residues used as raw materials in the process according to the invention contain impurities which are further concentrated in the product in the process described here and interfere with the crystallization process. In this case, other process steps, such as flashing of the excess alcohol, catalyst removal and glycerol removal, may optionally be carried out. These optional process steps are described below.

### Follow-up Processes before Crystallization of the Sterols “Flashing” of the Excess Alcohol (II)

In the “flashing of the excess alcohol (II)” process step, the hot reaction mixture from the “transesterification of the sterol esters” is expanded into a receiver, 55 to 85% of the excess alcohol distilling off. The system cools down considerably—to 75-85° C. where methanol is used. The residual alcohol still left in the reaction product is preferably not distilled off and serves as solubilizer in the following stage.

### Catalyst Removal (II)

The catalyst used in the transesterification of the sterol esters is soluble in the reaction mixture. In order to be able to remove the catalyst, it is converted with acids into an insoluble compound and precipitated as described in EP 0 656 894 B1. After precipitation, the salt precipitated is removed. In order to achieve separation of the organic from the aqueous phase, 30 to 200% by weight and preferably 50 to 100% by weight of fatty acid methyl ester, based on the amount of product used in the transesterification of the sterol esters, is added to the mixture in accordance with the invention.

If the transesterification of the sterol esters is carried out under pressure in the presence of a metal soap as catalyst, the precipitated metal may alternatively be adsorbed. In this case, all the alcohol may be removed in the preceding “flashing of the excess alcohol (II)” process step. Suitable adsorbents are amorphous silica gels charged with organic acids such as, for example, Trisyl types (Grace).

### Crystallization of the Sterols

The free sterols may then be purified by crystallization. Successful crystallization typically requires a free sterol concentration of at least 20 to 25%. Sterol concentrations of >40% can be achieved by the process according to the invention. Should the concentration still be below a value which does not allow reasonable crystallization, it is increased by distilling off the fatty acid esters produced in the “transesterification of the sterol esters” process step. The procedure involved corresponds to the “fatty acid ester distillation” step.

If the transesterification of the sterol esters was carried out under pressure and the metal soaps precipitated were removed by adsorption, fatty acid methyl ester (FME) is added as solvent. In this case, the quantity of FME is again 30 to 200% by weight and preferably 50 to 100% by weight, based on the amount of product used in the transesterification of the sterol esters.

For crystallization, the mixture is first heated in accordance with the invention to a temperature of 40 to 120° C. and preferably to a temperature of 50 to 90° C. and then cooled to 20° C. Cooling may optionally be accompanied by stirring. At sterol concentrations of more than 25%, complete solidification of the suspension can be avoided by stirring.

According to the invention, in order to increase the sterol yield, part of the mother liquor is recycled, for example to the crystallization process, after filtration of the crystal suspension. The return stream is fed to the system together with the fatty acid esters in the “catalyst removal (II)” process step. Another way of recycling the mother liquor is to introduce it into the first (a) or second (e) transesterification step.

The recycle ratio of the mother liquor depends to a very large extent on the starting material and hence on the composition of the mother liquor. It may be in the range from 0.1 to 5.0. A recycle ratio of 0.2 to 3.0 is preferably established.

### Crystallizate Washing

The crystals produced are washed with suitable solvents and then dried. Very light sterol crystals with a purity of more than 95% are obtained.

The present invention will now be illustrated in more detail by reference to the following specific, non-limiting examples.

### EXAMPLE 1

2.5 kg of residue from the distillation of transesterified palm kernel oil with a residual acid value of 3.5 are transesterified with 375 g (=15%) of methanol in the presence of 37.5 g (=1.5%) of sodium methylate at 122° C. A pressure of 5 bar builds up. After 8 minutes, the reaction mixture is drained off into a glass flask into which 58 g (=2.3%) of 50% citric acid solution have been introduced. 80% of the excess methanol are “flashed off” and at the same time the catalyst is neutralized. The mixture cools down to 75° C.

After stirring for 15 minutes, 250 g of water are added and stirring is continued for another 60 minutes at 75° C. The mixture is then cooled and the aqueous phase is drained off. The organic phase is washed twice with 250 g of water.

To remove the methyl ester, the product is distilled in a thin-layer evaporator at 180° C./3 mbar. The feed is run in at 90° C. The temperature of the condenser is 50° C. A ratio of distillate to bottom product of 75:25 is obtained for a throughput of 150 g/min. The methyl ester yield is thus 70%, based on the residue from the distillation of transesterified palm kernel oil.

130 g of the bottom product from the ME distillation are transesterified by addition of 65 g (=50%) of methanol and 2.6 g (=2.0%) of Na methylate at 120° C. After 5 h, the reaction is stopped by addition of 4.0 g (=3.1%) of 50% citric acid and the excess methanol is flashed off. The mixture cools to 75° C.

After stirring for 15 mins., 13 g of water are added to the reaction mixture and, after stirring for another 30 mins. at 75° C., 110 g of fatty acid methyl ester are added to facilitate phase separation. Phase separation takes place at 60° C. After separation of the aqueous phase, the organic phase is washed with 39 g of water.

a) The organic phase is heated to 65° C., after which the stirrer and the heating are switched off. After 60 mins., the mixture cools down to 25° C. and the maximum possible amount of crystallizate is obtained.

b) The organic phase is heated to 65° C. and then poured into an unheated vessel. After 25 mins., the mixture cools down to below 30° C. and the maximum possible amount of crystallizate is obtained.

c) The organic phase is heated to 65° C. and cooled with stirring to 20° C. at a rate of 0.5° C./min. Sterol crystals are obtained, but in a much smaller quantity than in Examples 1a) and 1b).

The crystals obtained in Examples 1a) and 1b) are washed with suitable solvents. After drying, sterols are obtained in a yield of 15.5 g, corresponding to 42.7%, based on the total sterol content of the residue from the distillation of transesterified palm kernel oil. The sterol concentration in the end product is >95%.

### EXAMPLE 2

The procedure described in Example 1 is repeated up to and including transesterification of the sterol esters. In the subsequent washing step, 20% of the mother liquor obtained in Example 1b) is also added to the mixture in addition to the 110 g of fatty acid methyl ester. All other steps are carried out as in Example 1, crystallization being carried out as in Example 1b).

By recycling 20% of the mother liquor, the sterol yield can be increased to 19 g and hence to 52.3%, based on the total sterol content of the residue from the distillation of transesterified palm kernel oil. The sterol concentration in the end product is >95%.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A process for producing sterols, said process comprising:
(a) providing an oil distillation residue, said residue comprising sterol esters and partial glycerides;
(b) transesterifying the partial glycerides with a lower alcohol in the presence of a basic catalyst under mild transesterification conditions to form fatty acid alkyl esters and glycerol;
(c) removing excess lower alcohol, the basic catalyst, the glycerol and the fatty acid alkyl esters, to form a bottom product comprising the sterol esters; and
(d) transesterifying the sterol esters at a temperature of from 90° C. to 145° C. and a pressure of from 2 to 10 bar for a period of from 4 to 10 hours to form free sterols.

2. The process according to claim 1, wherein the oil distillation residue is vegetable oil-derived.

3. The process according to claim 1, wherein the oil distillation residue comprises a residue derived from an oil selected from the group consisting of soybean oil, sunflower oil, rapeseed oil, coconut oil, palm oil, palm kernel oil, and mixtures thereof.

4. The process according to claim 1, wherein the transesterification of the partial glycerides under mild conditions is carried out at a temperature of from 115° C. to 145° C. and a pressure of from 2 to 10 bar, for a period of from 5 to 20 minutes.

5. The process according to claim 1, wherein the transesterification of the partial glycerides under mild conditions is carried out at a temperature of from 120° C. to 130° C. and a pressure of from 2 to 10 bar, for a period of from 8 to 15 minutes.

6. The process according to claim 1, wherein the lower alcohol comprises methanol.

7. The process according to claim 1, wherein removing excess lower alcohol comprises allowing expansion until the reaction temperature has cooled to a temperature of from 65° C. to 85° C.

8. The process according to claim 4, wherein the lower alcohol comprises methanol.

9. The process according to claim 4, wherein removing excess lower alcohol comprises allowing expansion until the reaction temperature has cooled to a temperature of from 65° C. to 85° C.

10. The process according to claim 1, wherein the removal of the basic catalyst comprises the addition of an aqueous solution of an acid, precipitation of the catalyst and separation of the precipitate.

11. The process according to claim 1, wherein the fatty acid alkyl esters are removed by distillation.

12. The process according to claim 11, wherein the distillation is carried out at a temperature of from 170° C. to 200° C. and a pressure of from 1 to 5 mbar.

13. The process according to claim 4, wherein the fatty acid alkyl esters are removed by distillation.

14. The process according to claim 13, wherein the distillation is carried out at a temperature of from 170° C. to 200° C. and a pressure of from 1 to 5 mbar.

15. The process according to claim 8, wherein the fatty acid alkyl esters are removed by distillation.

16. The process according to claim 15, wherein the distillation is carried out at a temperature of from 170° C. to 200° C. and a pressure of from 1 to 5 mbar.

17. The process according to claim 1, wherein the transesterification of the sterol esters is carried out at a temperature of from 120° C. to 130° C. and at a pressure of from 2 to 10 bar, for a period of from 5 to 8 hours.

18. The process according to claim 1, further comprising purification of the free sterols via crystallization and washing.

19. A process for producing sterols, said process comprising:
(a) providing an oil distillation residue derived from an oil selected from the group consisting of soybean oil, sunflower oil, rapeseed oil, coconut oil, palm oil, palm kernel oil, and mixtures thereof, said residue comprising sterol esters and partial glycerides;
(b) transesterifying the partial glycerides and the free fatty acid based partial glycerides with methanol in the presence of a basic catalyst at a temperature of from 115° C. to 145° C. and a pressure of from 2 to 10 bar, for a period of from 5 to 20 minutes, to form fatty acid methyl esters and glycerol;
(c) removing excess methanol, the basic catalyst, and the glycerol, and distilling off the fatty acid methyl esters at a temperature of from 170° C. to 200° C. and a pressure of from 1 to 5 mbar, to form a bottom product comprising the sterol esters; and
(e) transesterifying the sterol esters at a temperature of from 90° C. to 145° C. and a pressure of from 2 to 10 bar for a period of from 4 to 10 hours to form free sterols.
